(19) United States
(12) Patent Application Publication
(10) Pub. No.: US 2003/0032586 A1
(43) Pub. Date: Feb. 13, 2003

(54) COMPOSITIONS FOR MORPHOGEN-INDUCED OSTEOGENESIS

(76) Inventors: DAVID C. RUEGER, SOUTHBOROUGH, MA (US); MARJORIE M. TUCKER, HOLLISTON, MA (US)

Correspondence Address: JAMES F. HALEY, FISH & NEAVE, 1251 AVENUE OF THE AMERICAS, NEW YORK, NY, 100201104

(21) Appl. No.: 09/039,107

(22) Filed: Mar. 14, 1998

Related U.S. Application Data
(60) Provisional application No. 60/046,589, filed on May 15, 1997.

(51) Int. Cl. A61K 38/00; A01N 37/18

(52) U.S. Cl. 514/12; 514/2

(57) ABSTRACT

Disclosed herein are improved osteogenic devices and methods of use thereof for repair of bone and cartilage defects.

### FIELD OF THE INVENTION

The invention relates to materials and methods for correcting orthopedic defects using osteogenic proteins.

### BACKGROUND OF THE INVENTION

Morphogens are able to induce the proliferation and differentiation of progenitor cells into functional bone, cartilage, tendon, and/or ligament tissue. This class of proteins includes members of the family of bone morphogenetic proteins (BMPs) identified by their ability to induce ectopic, endochondral bone morphogenesis. The morphogens, also referred to as, osteogenic proteins generally are classified as a subgroup of the TGF-β superfamily of growth factors (Hogan (1996) Genes & Development 10:1580-1594). Members of the morphogen family of proteins include the mammalian osteogenic protein-1 (OP-1, also known as BMP-7, and the Drosophila homolog 60A), osteogenic protein-2 (OP-2, also known as BMP-8), osteogenic protein-3 (OP-3), BMP-2 (also known as BMP-2A or CBMP-2A, and the Drosophila homolog DPP), BMP-3, BMP-4 (also known as BMP-2B or CBMP-2B), BMP-5, BMP-6 and its murine homolog Vgr-1, BMP-9, BMP-10, BMP-11, BMP-12, GDF3 (also known as Vgr2), GDF8 , GDF9, GDF10, GDF11, GDF12, BMP-13, BMP-14, BMP-15, GDF-5 (also known as CDMP-1 or MP52), GDF-6 (also known as CDMP-2), GDF-7 (also known as CDMP-3), the Xenopus homolog Vg1 and NODAL, UNIVIN, SCREW, ADMP, and NEURAL.

Members of this family encode secreted polypeptides that share common structural features. The mature form of such proteins results from processing through a “pro-form” to yield a mature polypeptide chain competent to dimerize and containing a carboxy terminal active domain of approximately 97-106 amino acids. All members share a conserved pattern of cysteines in this domain and the active form of these proteins can be either a disulfide-bonded homodimer of a single family member or a heterodimer of two different members (see, e.g., Massague (1990) Annu. Rev. Cell Biol. 6:597; Sampath, et al. (1990) J. Biol. Chem. 265:13198). See also, U.S. Pat. No. 5,011,691; U.S. Pat. No. 5,266,683, Ozkaynak et al. (1990) EMBO J. 9: 2085-2093, Wharton et al. (1991) PNAS 88:9214-9218), (Ozkaynak (1992) J. Biol. Chem. 267:25220-25227 and U.S. Pat. No. 5,266,683); (Celeste et al. (1991) PNAS 87:9843-9847); (Lyons et al. (1989) PNAS 86:4554-4558). These disclosures describe the amino acid and DNA sequences, as well as the chemical and physical characteristics, of osteogenic proteins. See also, Wozney et al. (1988) Science 242:1528-1534); BMP 9 (WO93/00432, published Jan. 7, 1993); DPP (Padgett et al. (1987) Nature 325:81-84; and Vg-1 (Weeks (1987) Cell 51:861-867).

True osteogenic proteins capable of inducing the above-described cascade of morphogenic events resulting in endochondral bone formation, have now been identified, isolated, and cloned. Whether naturally-occurring or synthetically prepared, these osteogenic factors, when implanted in a mammal in association with a matrix or substrate that allows attachment, proliferation and differentiation of migratory progenitor cells, can induce recruitment of accessible progenitor cells and stimulate their proliferation, thereby inducing differentiation into chondrocytes and osteoblasts, and further inducing differentiation of intermediate cartilage, vascularization, bone formation, remodeling, and, finally, marrow differentiation. Furthermore, numerous practitioners have demonstrated the ability of these osteogenic proteins, when admixed with either naturally-sourced matrix materials such as collagen or synthetically-prepared polymeric matrix materials, to induce bone formation, including endochondral bone formation, under conditions where true replacement bone otherwise would not occur. For example, when combined with a matrix material, these osteogenic proteins induce formation of new bone in large segmental bone defects, spinal fusions, and fractures.

Needs remain for carriers for delivering osteogenic protein to a bone repair locus. Preferred carriers are provided by the present invention.

### SUMMARY OF THE INVENTION

The present invention provides delivery systems and methods for providing osteogenic protein to bone defect sites. In a preferred emodiment, a delivery system of the invention comprises osteogenic protein in a calcium phosphate matrix. It has now been recognized that use of a calcium phosphate matrix for delivery of osteogenic protein to defect sites in bones that do not undergo active remodeling, especially in short, irregular, or flat bone defect sites, greatly improves the structure of new bone ingrowth. It has also been recognized that providing preferred ratios of calcium phosphate to hydroxyapatite results in improved cosmetic results.

The invention provides, in one aspect, a device for inducing local bone and/or cartilage formation. A preferred device of the invention comprises an osteogenic protein in a calcium phosphate matrix. As contemplated herein, the device preferably comprises osteogenic proteins such as, but not limited to, OP-1, OP-2, BMP-2, BMP-4, BMP-5 and BMP-6. A currently preferred osteogenic protein is OP-1. As used herein, the terms “morphogen”, “bone morphogen”, “bone morphogenic protein”, “BMP”, “osteogenic protein” and “osteogenic factor” embrace the class of proteins typified by human osteogenic protein 1 (hOP-1). Nucleotide and amino acid sequences for hOP-1 are provided in Seq. ID Nos. 1 and 2, respectively. OP-1 is merely representative of the TGF-β subclass of true chondrogenic tissue morphogens competent to act as osteogenic proteins, and is not intended to be limiting. Other known, and useful proteins include, BMP-2, BMP-3, BMP-3b, BMP-4, BMP-5, BMP-6, BMP-8, BMP-9, BMP-10, BMP-11, BMP-12, BMP-13, BMP-15, GDF-1, GDF-2, GDF-3, GDF-5, GDF-6, GDF-7, GDF-8, GDF-9, GDF-10, GDF-11, GDF-12, NODAL, UNIVIN, SCREW, ADMP, NEURAL and osteogenically active amino acid variants thereof. In one preferred embodiment, proteins useful in the invention include biologically active species variants of any of these proteins, including conservative amino acid sequence variants, proteins encoded by degenerate nucleotide sequence variants, and osteogenically active proteins sharing the conserved seven cysteine skeleton as defined herein and encoded by a DNA sequence competent to hybridize to a DNA sequence encoding an osteogenic protein disclosed herein, including, without limitation, OP-1, BMP-5, BMP-6, BMP-2, BMP-4 or GDF-5, GDF-6 or GDF-7. In another embodiment, useful osteogenic proteins include those sharing the conserved seven cysteine domain and sharing at least 70% amino acid sequence homology (similarity) within the C-terminal active domain, as defined herein. In another embodiment, useful proteins include those sharing greater than 60% identity in the C-terminal domain. In still another embodiment, useful osteogenic proteins can be defined as osteogenically active proteins having any one of the generic sequences defined herein, including OPX (SEQ ID No: 3) and Generic Sequences 7 and 8 (Seq. ID Nos. 4 and 5), or Generic Sequences 9 and 10 (Seq. ID Nos. 6 and 7).

A calcium phosphate matrix for use in the invention may be supplied in any biocompatible form, and is preferably in the form of hydroxyapatite, tricalcium phosphate, or any other form that is resorbable during new bone growth. Calcium phoshpate may be supplied in the form of a powder, solid blocks, cements, pastes, shaped forms, or any other form that is resorbed, in whole or in part, during new bone ingrowth. Calcium phosphate for use in a device or method of the invention may be integrated or admixed with other carrier materials, such as collagen or a collagen-based carrier, including polyglycolic acid, polylactic acid, polybutyric acid, polysylicates, and their derivatives, or combinations.

In another aspect, the instant invention provides methods for inducing local bone or cartilage formation, or for repair of bone, cartilage or osteochondral defects. In a preferred embodiment, a method of the invention comprises administering to a bone defect site an osteogenic protein in a calcium phosphate matrix. It has now been discovered that a calcium phosphate matrix is superior to other matrices for repair of defects, especially in short, irregular, or flat bones. Accordingly, in a particularly preferred embodiment, the invention provides a method for inducing new bone growth in a small bone of the head, face, hands, or feet, comprising implanting an osteogenic device in a short, irregular, or flat bone defect site, the osteogenic device comprising an osteogenic protein in a calcium phosphate matrix in an amount sufficient to stimulate new bone growth in the defect site. Methods of the invention are particularly useful for providing augmentation of the bones of the face, such as, for example, the mandible or the maxilla. While a calcium phosphate matrix is useful for reconstruction of any bone, including long bones, methods of the invention are particularly useful in cosmetic surgery procedures, wherein precise bone reconstruction is desirable or required.

Also in a preferred embodiment, the invention provides methods for repairing a bone defect comprising filling the defect with a composition comprising a morphogen in combination with an allograft material, preferably allograft bone chips. In highly preferred embodiments, the invention comprises inserting an impacted allograft into a defect site in order to improve mechanical fixation of the implant.

The instant methods are useful to induce formation of at least endochondral bone, intramembranous bone, and articular cartilage. Bone repair methods of the invention include treatment of both closed and open defects with the above-described improved osteogenic devices. As taught herein, the methods of the instant invention can be practiced using improved devices that are of sufficient volume to fill the defect site, as well as using improved devices that are not. Examples of defects include, but are not limited to, critical size defects, non-critical size defects, non-union fractures, fractures, osteochondral defects, chondral defects and periodontal defects. Further aspects and advantage of the invention will be appreciated upon consideration of the following detailed description thereof.

### BRIEF DESCRIPTION OF THE DRAWINGS

FIG. 1 (panels 1A through 1L) is a tabular alignment of the amino acid sequences of various naturally occurring morphogens with a preferred reference sequence of human OP1, residues 330-431 of SEQ ID NO: 1;

FIG. 2 is a tabular presentation of alternative amino acids for “Xaa” positions in generic sequences SEQ ID NOS: 4, 5, and 8 that represent amino acid variations in known morphogens;

FIG. 3 is a tabular presentation of alternative amino acids for “Xaa” positions in generic sequences SEQ ID NOS: 6, 7, and 9 that represent amino acid variations in known morphogens;

FIG. 4 is a tabular presentation of alternative amino acids for “Xaa” positions in generic sequence SEQ ID NO: 3 that represents amino acid variations in several identified allelic and phylogenetic variants of OP-1 and OP-2.

### DETAILED DESCRIPTION OF PREFERRED EMBODIMENTS

Bones are generally divided into four main types. Long bones are the largest bones of the body (e.g., the femur); short bones are shorter than long bones, and have less prominent ends (e.g., bones of the hands and feet); irregular bones typically possess surfaces that articulate with other bones (e.g., wrist bones); and flat bones typically have plate-like surfaces (e.g., bones of the skull). It has now been discovered that bone repair is greatly facilitated by application to a defect site of an osteogenic protein in a calcium phosphate matrix. It has also been discovered that the use of an optimal ratio of calcium phosphate to osteogenic protein promotes optimal cosmetic bone ingrowth. These effects are most prominent in short, irregular and flat bones, especially where optimal cosmetic results are desirable.

As used herin, “defect”, “defect site”, or “defect locus”, defines an orthopedic structural disruption requiring repair. The defect may occur in a joint, in any bone, including a intramembranous bone, bony, cartilage, tendon, ligament, or an osteochondral defect. A defect can be the result of accident, disease, surgical manipulation, and/or prosthetic failure. In certain embodiments, the defect is a void having a volume incapable of endogenous or spontaneous repair. Such defects are generally twice the diameter of the subject bone and are also called “critical size” defects. For example, in rabbit and monkey segmental defect models, the gap is approximately 1.5 cm and 2.0 cm, respectively. In a canine ulnar defect model, the defect is a 2-4 cm, gap incapable of spontaneous repair. See, e.g., Schmitz et al., Clinical Orthopaedics and Related Research 205:299-308 (1986); Vukicevic et al., in Advanced in Molecular and Cell Biology, Vol. 6, pp. 207-224 (1993)(JAI Press, Inc.). In other embodiments, the defect is a non-critical size segmental defect. Generally, non-critical defects, such as fracture defects, are capable of some spontaneous repair. Application of the devices and formulations described herein can substantially enhance fracture repair, including the rate and quality of newly formed bone. This allows for improved bone healing, especially in compromised individuals such as diabetics, smokers, obese individuals and others who, due to an acquired or congenital condition have a reduced capacity to heal bone fractures. Other defects include osteochondral defect, such as osteochondral plugs. Such a defect traverses the entirety of the overlying cartilage and enters, at least in part, the underlying bony structure. In contrast, a chondral or subchondral defect traverses the overlying cartilage, in part or in whole, respectively, but does not involve the underlying bone. Other orthopedic defects susceptible to repair using the instant invention include, but are not limited to, non-union fractures; bone cavities; tumor resection; fresh fractures (distracted or undistracted); cranial/facial abnormalities; periodontal defects and irregularities; spinal fusions; as well as those defects resulting from diseases such as cancer, arthritis, including osteoarthritis, and other bone degenerative disorders such as osteochondritis dessicans. Still other defects susceptible to repair include joint tissue defects, including defects requiring partial or complete joint reconstruction, including correcting tendon and/or ligamentous tissue defects such as, for example, the anterior, posterior, lateral and medial ligaments of the knee, the patella and achilles tendons, and the like.

In addition to osteogenic proteins, various growth factors, hormones, enzymes, therapeutic compositions, antibiotics, or other bioactive agents also can be contained within an osteogenic device. Thus, various known growth factors such as EGF, PDGF, IGF, FGF, TGF-a, and TGF-β can be combined with an osteogenic device and be delivered to the defect locus. An osteogenic device also can be used to deliver chemotherapeutic agents, insulin, enzymes, enzyme inhibitors and/or chemoattractant/chemotactic factors.

The means for making and using the methods, implants and devices of the invention, as well as other material aspects concerning their nature and utility, including how to make and how to use the subject matter claimed, will be further understood from the following, which constitutes the best mode currently contemplated for practicing the invention. It will be appreciated that the invention is not limited to such exemplary work or to the specific details set forth in these examples.

### I. PROTEIN CONSIDERATIONS

A. Biochemical, Structural and Functional Properties of Bone Morphogenic Proteins

In its mature, native form, natural-sourced osteogenic protein is a glycosylated dimer, typically having an apparent molecular weight of about 30-36 kDa as determined by SDS-PAGE. When reduced, the 30 kDa protein gives rise to two glycosylated peptide subunits having apparent molecular weights of about 16 kDa and 18 kDa. In the reduced state, the protein has no detectable osteogenic activity. The unglycosylated protein, which also has osteogenic activity, has an apparent molecular weight of about 27 kDa. When reduced, the 27 kDa protein gives rise to two unglycosylated polypeptide chains, having molecular weights of about 14 kDa to 16 kDa. Typically, the naturally occurring osteogenic proteins are translated as a precursor, having an N-terminal signal peptide sequence typically less than about 30 residues, followed by a “pro” domain that is cleaved to yield the mature C-terminal domain. The signal peptide is cleaved rapidly upon translation, at a cleavage site that can be predicted in a given sequence using the method of Von Heijne, NUCLEIC ACIDS RESEARCH 14: 4683-4691 (1986). The pro domain typically is about three times larger than the fully processed mature C-terminal domain.

Osteogenic proteins useful herein include any known naturally-occurring native proteins including allelic, phylogenetic counterpart and other variants thereof, whether naturally-occurring or biosynthetically produced (e.g., including “muteins” or “mutant proteins”), as well as new, osteogenically active members of the general morphogenic family of proteins.

Particularly useful sequences include those comprising the C-terminal 96 or 102 amino acid sequences of DPP (from Drosophila), Vg1 (from Xenopus), Vgr-1 (from mouse), the OP-1 and OP-2 proteins (see U.S. Pat. No. 5,011,691 and Oppermann et al., as well as the proteins referred to as BMP2, BMP3, BMP4 (see WO88/00205, U.S. Pat. No. 5,013,649 and WO91/18098), BMP5 and BMP6 (see WO90/11366, PCT/US90/01630), BMP8 and BMP9. Other proteins useful in the practice of the invention include active forms of OP1, OP2, OP3, BMP2, BMP3, BMP4, BMP5, BMP6, BMP9, GDF-5, GDF-6, GDF-7, DPP, Vg1, Vgr, 60A protein, GDF-1, GDF-3, GDF-5, GDF-6, GDF-7, BMP10, BMP11, BMP13, BMP15, UNIVIN, NODAL, SCREW, ADMP or NURAL and amino acid sequence variants thereof. In one currently preferred embodiment, osteogenic protein include any one of: OP1, OP2, OP3, BMP2, BMP4, BMP5, BMP6, BMP9, and amino acid sequence variants and homologs thereof, including species homologs, thereof. Publications disclosing OP-1 and OP-2 sequences, as well as their chemical and physical properties, include U.S. Pat. Nos. 5,011,691 and 5,266,683, incorporated by reference herein.

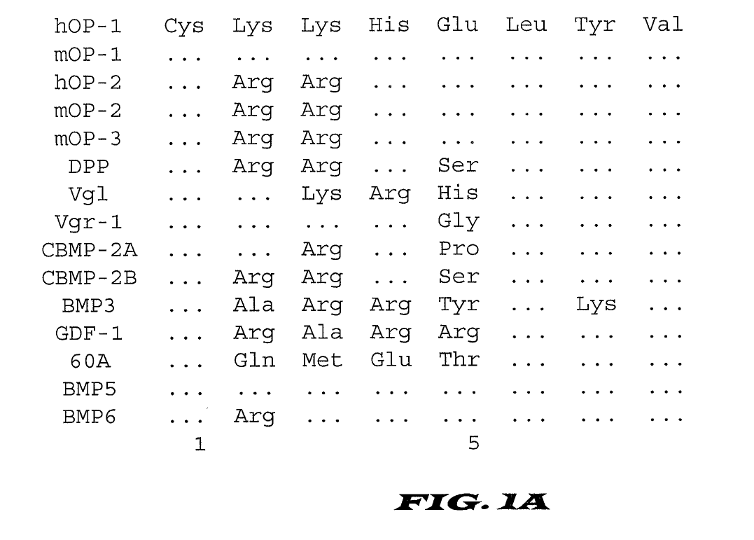

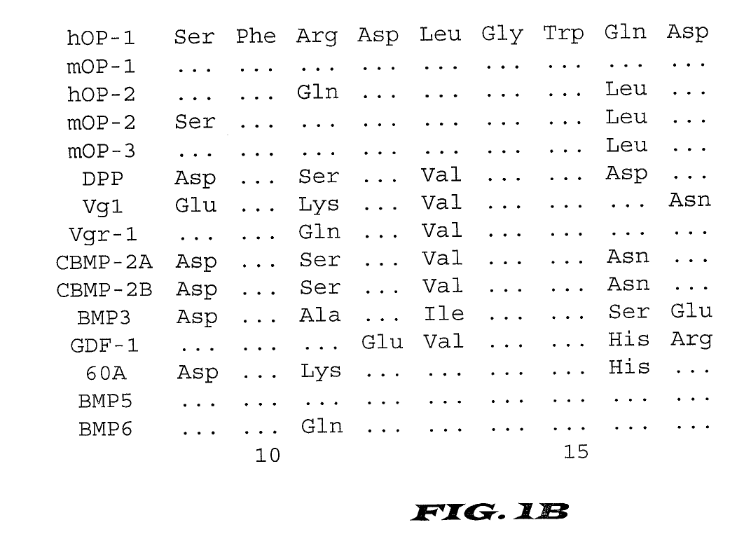

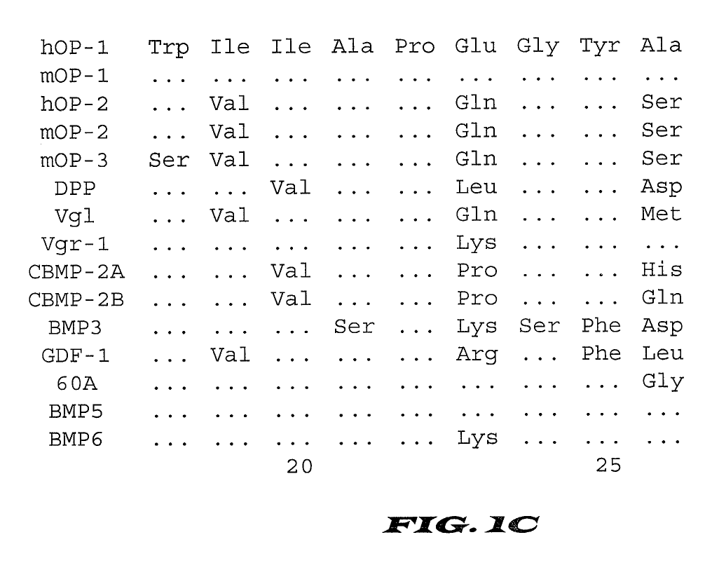

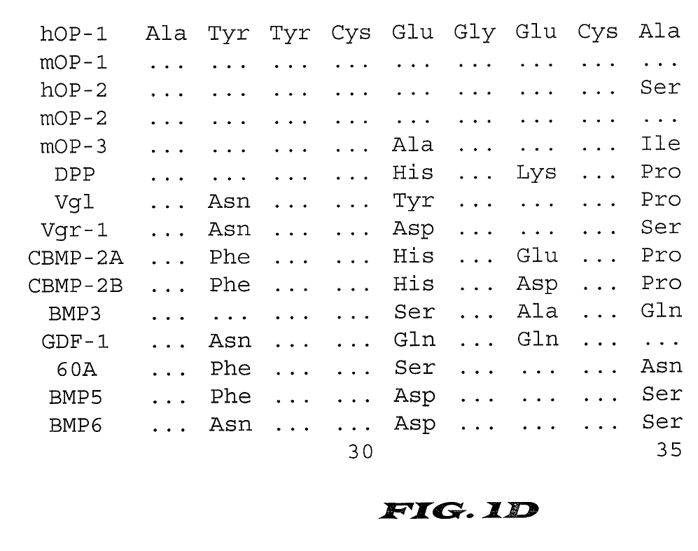

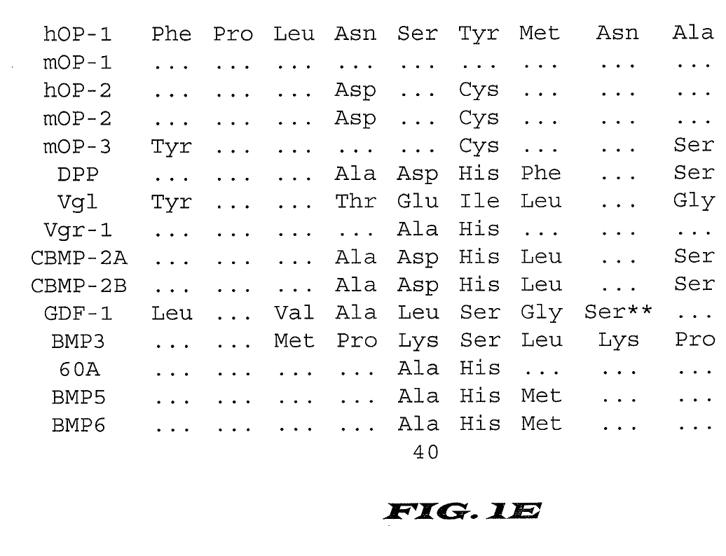

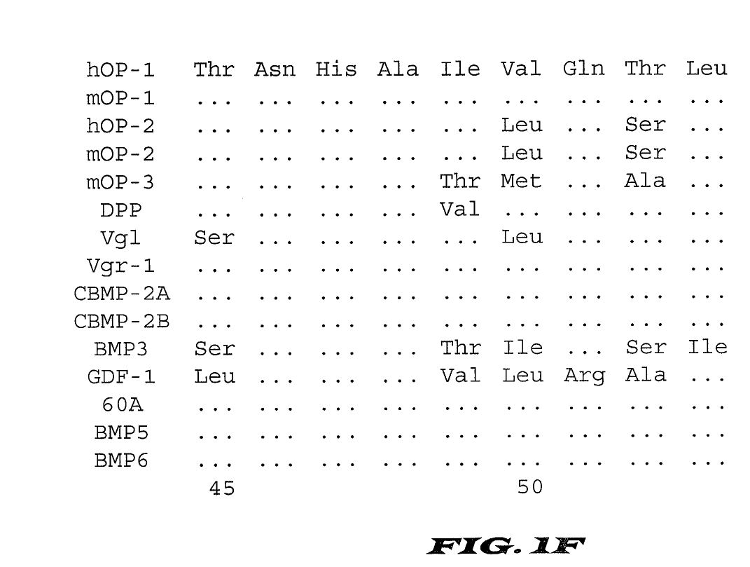

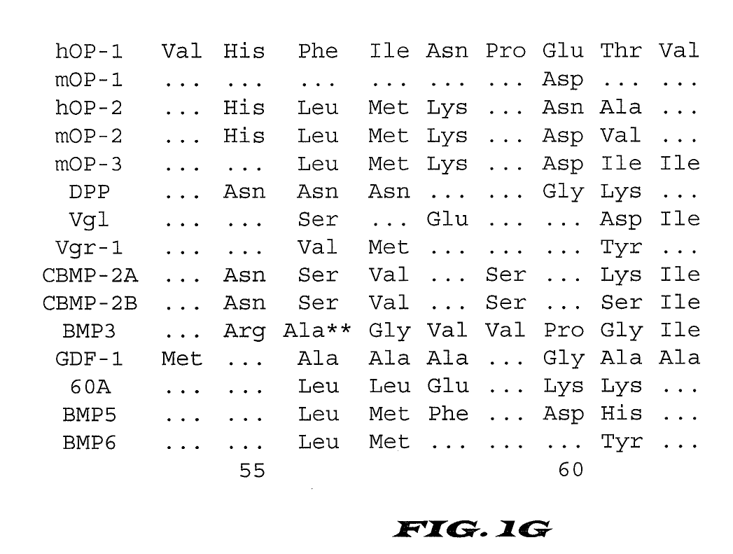

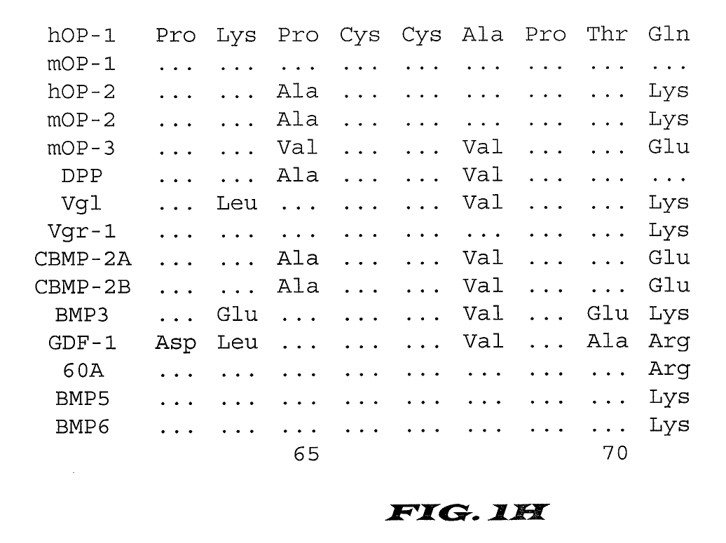

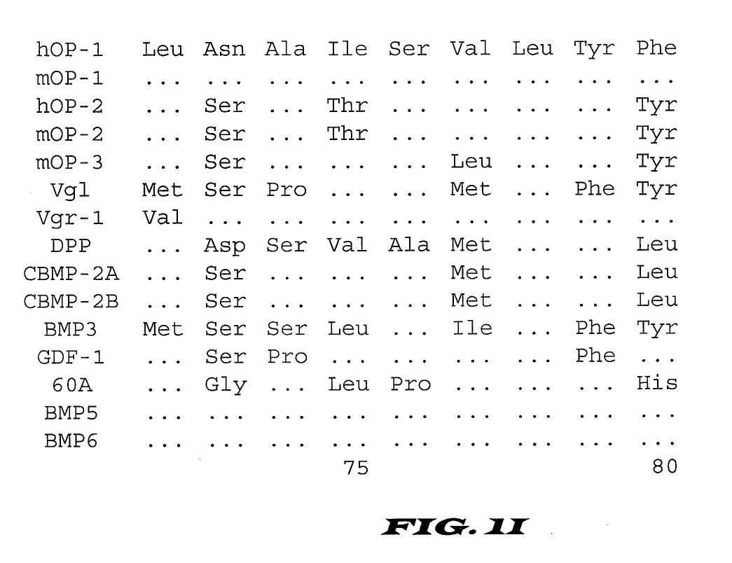

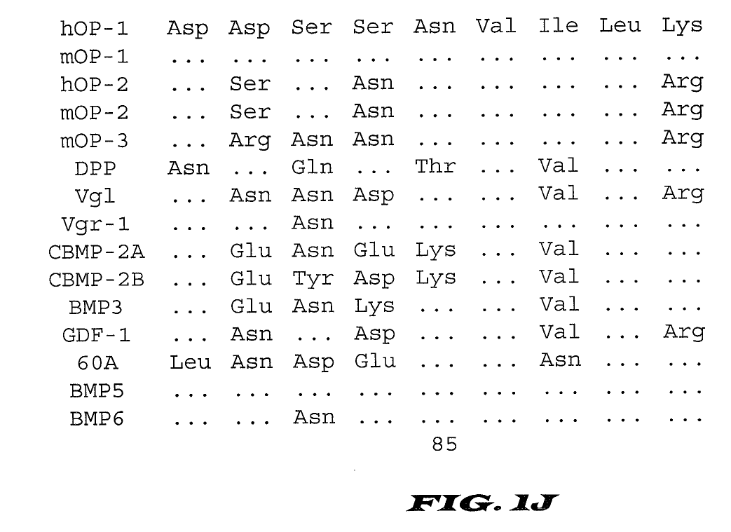

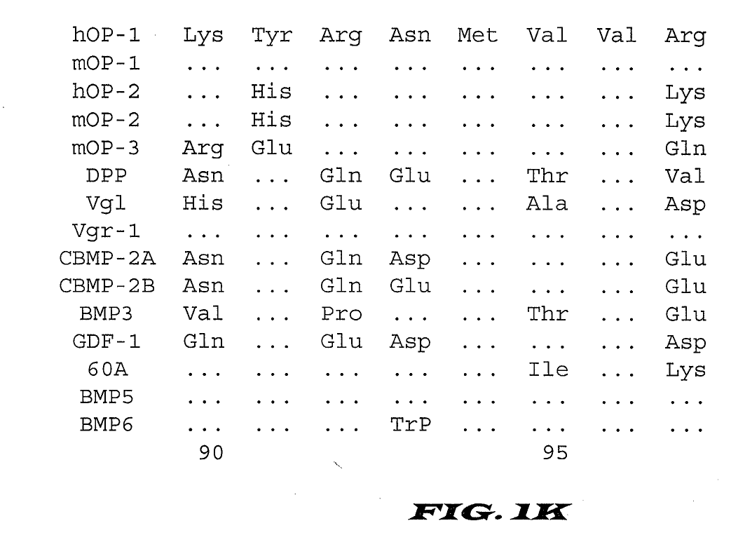

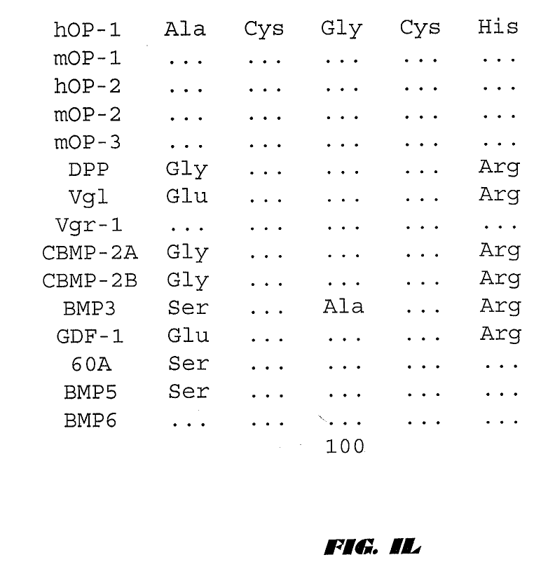

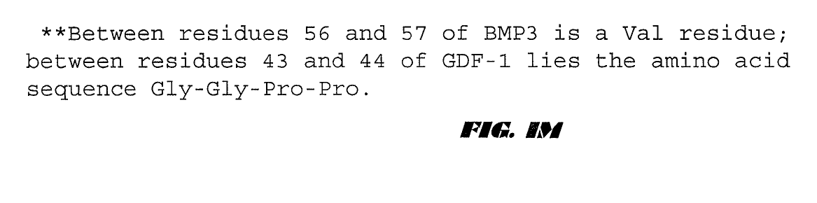

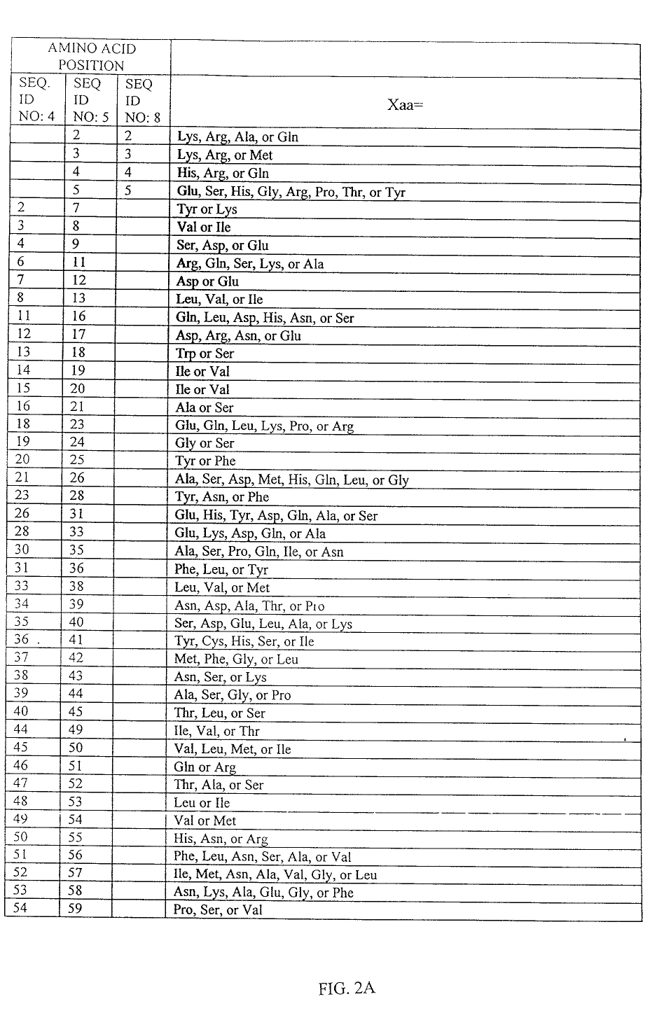

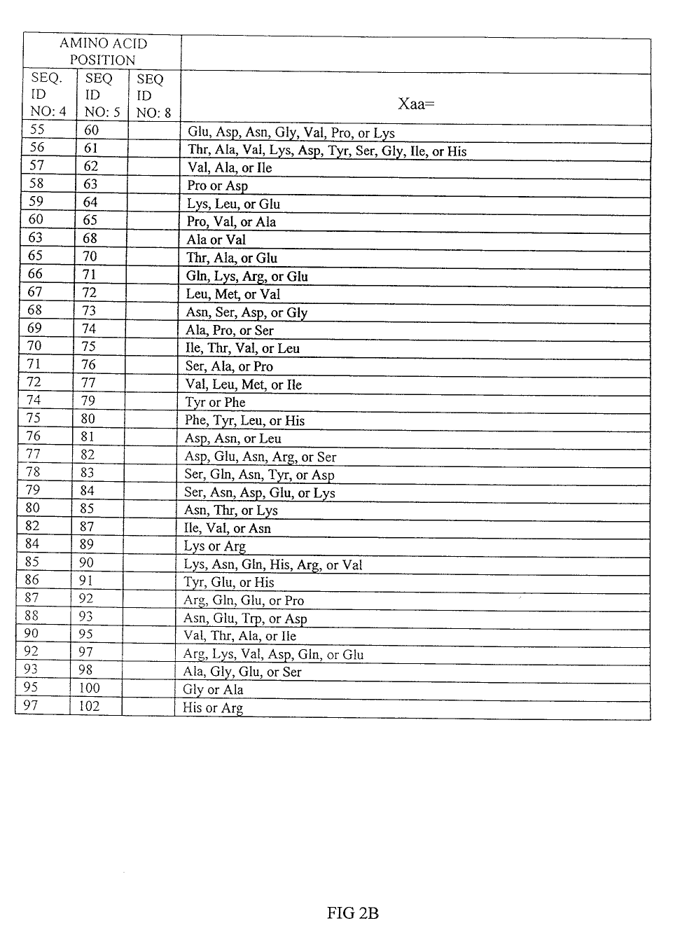

In preferred embodiments, morphogens for use in methods of the invention include proteins having at least 70% homology with the amino acid sequence of the C-terminal seven-cysteine skeleton of human OP-1, SEQ ID NO: 2, and having the ability to induce endochondral bone formation in the Reddi and Sampath assay described herein. Compounds that meet these requirements are considered functionally equivalent to a known response morphogen. To determine whether a candidate amino acid sequence is functionally equivalent to a reference morphogen, the candidate sequence and the reference sequence are aligned. The first step for performing an alignment is to use an alignment tool, such as the dynamic programming algorithm described in Needleman et al., J. MOL. BIOL. 48: 443 (1970), and the Align Program, a commercial software package produced by DNAstar, Inc. the teachings of which are incorporated by reference herein. After the initial alignment is made, it is then refined by comparison to a multiple sequence alignment of a family of related proteins, such as those shown in FIGS. 1A through 1M, which is a multiple sequence alignment of a family of known morphogens, including hOP-1. Once the alignment between the candidate and reference sequences is made and refined, a percent homology score is calculated. The individual amino acids of each sequence are compared sequentially according to their similarity to each other. Similarity factors include similar size, shape and electrical charge. One particularly preferred method of determining amino acid similarities is the PAM250 matrix described in Dayhoff et al., 5 ATLAS OF PROTEIN SEQUENCE AND STRUCTURE 345-352 (1978 & Supp.), incorporated by reference herein. A similarity score is first calculated as the sum of the aligned pairwise amino acid similarity scores. Insertions and deletions are ignored for the purposes of percent homology and identity. Accordingly, gap penalties are not used in this calculation. The raw score is then normalized by dividing it by the geometric mean of the scores of the candidate compound and the seven cysteine skeleton of hOP-1. The geometric mean is the square root of the product of these scores. The normalized raw score is the percent homology.

In an alternative preferred embodiment, a functionally-equivalent morphogen sequence shares at least 60% amino acid identity with a reference sequence. That is, any 60% of the aligned amino acids are identical to the corresponding amino acids in the reference sequence. Any one or more of the naturally-occurring or biosynthetic morphogens disclosed herein may be used as a reference sequence to determine whether a candidate sequence falls within the morphogen family. In a preferred embodiment, the reference sequence is the C-terminal seven-cysteine skeleton sequence of human OP-1 as shown in SEQ ID NO: 2. Examples of conservative substitutions for use in the above calculations include the substitution of one amino acid for another with similar characteristics, e.g., substitutions within the following groups are well-known: (a) valine, glycine; (b) glycine, alanine; (c) valine, isoleucine, leucine; (d) aspartic acid, glutamic acid; (e) asparagine, glutamine; (f) serine, threonine; (g) lysine, arginine, methionine; and (h) phenylalanine, tyrosine. The term “conservative variant” or “conservative variation” also includes the use of a substituted amino acid in place of an unsubstituted parent amino acid in a given polypeptide chain, provided that antibodies having binding specificity for the resulting substituted polypeptide chain also have binding specificity (i.e., “crossreact” or “immunoreact” with) the unsubstituted or parent polypeptide.

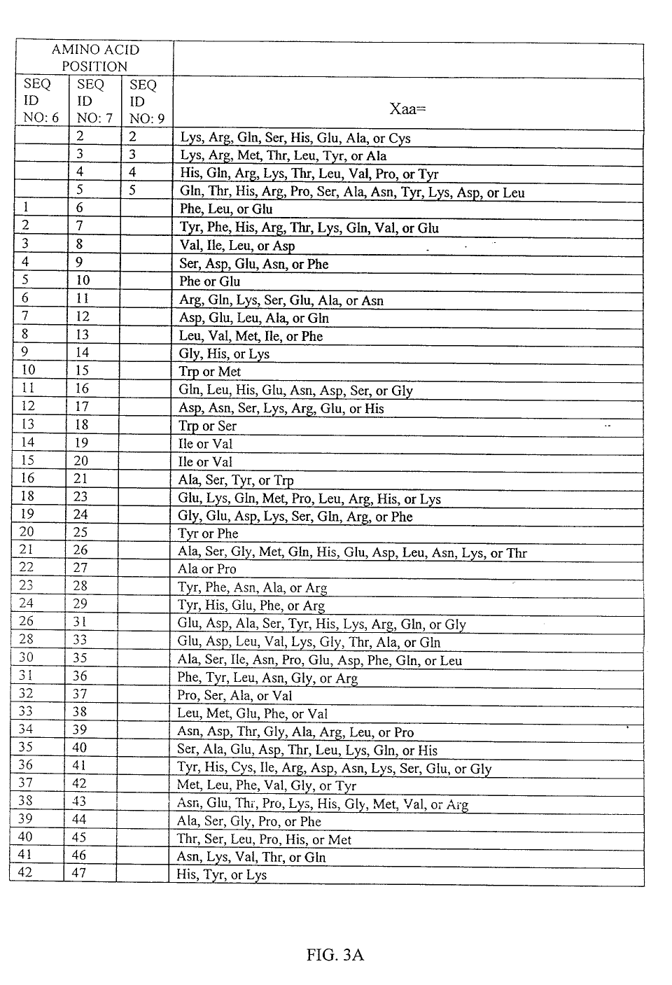

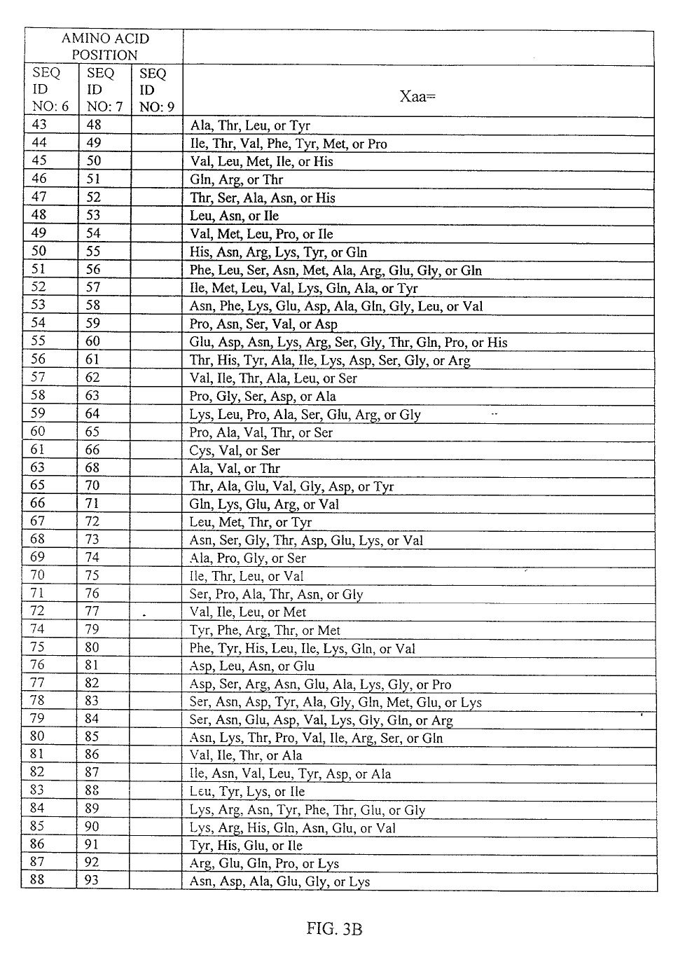

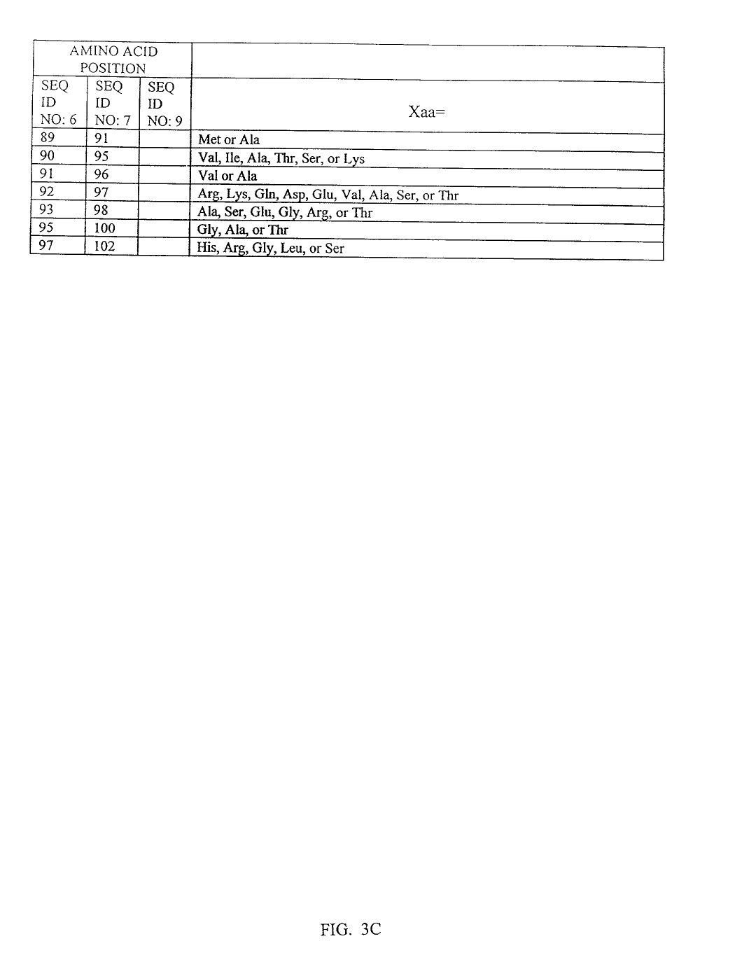

In a preferred embodiment, morphogens useful in the present invention are defined by a generic amino acid sequence that represents variations in known morphogens. For example, SEQ ID NOS: 4 and 5 encompass observed variations between preferred morphogens, including OP-1, OP-2, OP-3, CBMP-2A, CBMP-2B, BMP-3, 60A, DPP, Vg1, BMP-5, BMP-6, Vgr-1, and GDF-1. SEQ ID NO: 5 includes all of SEQ ID NO: 4, and also includes at its N-terminus the five amino acid sequence of SEQ ID NO: 8. The generic sequences include both the amino acid identity shared by these sequences in the C-terminal domain, defined by the six- and seven-cysteine skeletons (SEQ ID NOS: 4 and 5, respectively), and alternative amino acids for variable positions within the sequence. Positions that allow for alternative amino acids are represented by “Xaa”. FIG. 3 shows the alternative amino acids for each “Xaa” position in SEQ ID NOS: 4, 5 and 8. For example, referring to SEQ ID NO: 5 and FIG. 3, the “Xaa” at position 2 may be a tyrosine or a lysine. The generic sequences provide an appropriate cysteine skeleton for inter- or intramolecular disulfide bonding, and contain certain critical amino acids likely to influence the tertiary structure of the proteins. In addition, the “Xaa” at position 36 in SEQ ID NO: 4, or at position 41 in SEQ ID NO: 5, may be an additional cysteine, thereby encompassing the morphogenically-active sequences of OP-2 and OP-3.

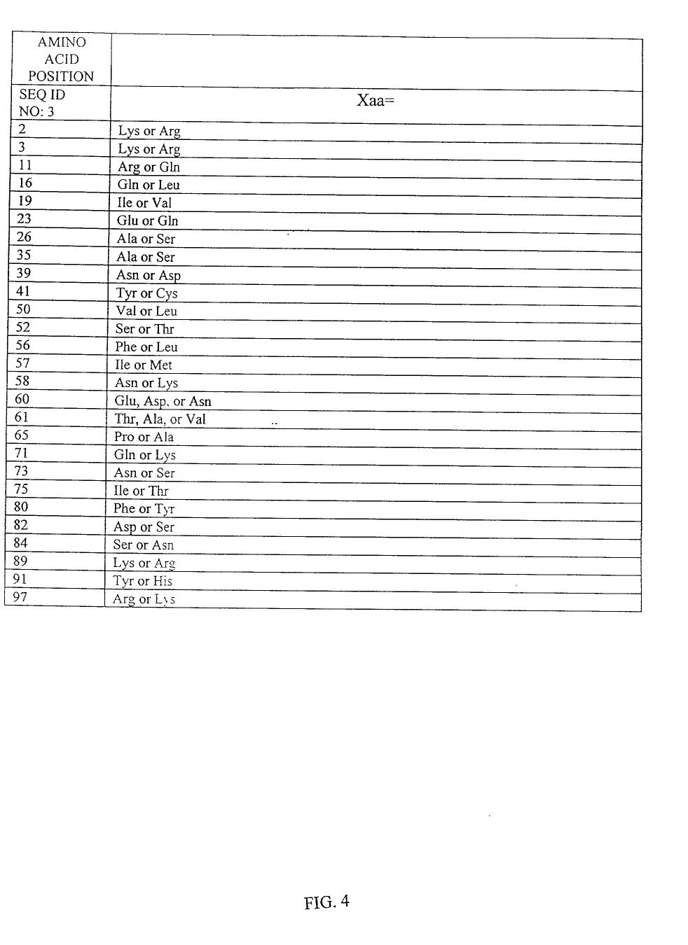

In another embodiment, useful morphogens include those defined by SEQ ID NOS: 6 or 7, which are composite amino acid sequences of the following morphogens: human OP-1, human OP-2, human OP-3, human BMP-2, human BMP-3, human BMP-4, human BMP-5, human BMP-6, human BMP-8, human BMP-9, human BMP-10, human BMP-11, Drosophila 60A, Xenopus Vg-1, sea urchin UNIVIN, human CDMP-1 (mouse GDF-5), human CDMP-2 (mouse GDF-6, human BMP-13), human CDMP-3 (mouse GDF-7, human BMP-12), mouse GDF-3, human GDF-1, mouse GDF-1, chicken DORSALIN, Drosophila dpp, Drosophila SCREW, mouse NODAL, mouse GDF-8, human GDF-8, mouse GDF-9, mouse GDF-10, human GDF-11, mouse GDF-11, human BMP-15, and rat BMP-3b. SEQ ID NO: 7 includes all of SEQ ID NO: 6 and also includes at its N-terminus the five amino acid sequence of SEQ ID NO: 9. SEQ ID NO: 6 accommodates the C-terminal six-cysteine skeleton, and SEQ ID NO: 7 accommodates the seven-cysteine skeleton. Positions that allow for alternative amino acids are represented by “Xaa”. FIG. 4 shows the alternative amino acids for each “Xaa” position in SEQ ID NOS: 6, 7 and 9.

As noted above, certain preferred morphogen sequences useful in this invention have greater than 60% identity, preferably greater than 65% identity, with the amino acid sequence defining the preferred reference sequence of hOP-1. These particularly preferred sequences include allelic and phylogenetic variants of the OP-1 and OP-2 proteins, including the Drosophila 60A protein, as well as the closely related proteins BMP-5, BMP-6 and Vgr-1. Accordingly, in certain particularly preferred embodiments, useful morphogens include proteins comprising the generic amino acid sequence SEQ ID NO: 3 (referred to herein as “OPX”), which defines the seven-cysteine skeleton and accommodates the homologies between several identified variants of OP-1 and OP-2. Positions that allow for alternative amino acids are represented by “Xaa”. FIG. 5 shows the alternative amino acids for each “Xaa” position in SEQ ID NO: 3.

In still another preferred embodiment, useful morphogens include those having an amino acid sequence encoded by a polynucleotide that hybridizes under high stringency conditions with DNA or RNA encoding a reference morphogen. Standard stringency conditions are well characterized in standard molecular biology texts. See generally MOLECULAR CLONING A LABORATORY MANUAL, (Sambrook et al., eds., 1989); DNA CLONING, Vol. I & II (D. N. Glover ed., 1985); OLIGONUCLEOTIDE SYNTHESIS (M. J. Gait ed., 1984); NUCLEIC ACID HYBRIDIZATION (B. D. Hames & S. J. Higgins eds. 1984); B. Perbal, A PRACTICAL GUIDE To MOLECULAR CLONING (1984).

In another embodiment, morphogens useful in the invention include the soluble complex form comprising a mature morphogen dimer linked to a morphogen pro domain or a solubility-enhancing fragment thereof. A solubility-enhancing fragment is any N-terminal or C-terminal fragment of a morphogen pro domain that forms a complex with the mature morphogen dimer and increases the solubility of the morphogen dimer. Preferably, the soluble complex comprises a morphogen dimer and two pro domain peptides. Morphogen soluble complex is described in published application WO 94/03600, incorporated by reference herein.

In yet another embodiment, useful morphogens include biologically active biosynthetic constructs, including novel biosynthetic morphogens and chimeric proteins designed using sequences from two or more known morphogens. See U.S. Pat. No. 5,011,691, incorporated by reference herein (e.g., COP-1, COP-3, COP-4, COP-5, COP-7, and COP-16).

### II. FORMULATION AND DELIVERY CONSIDERATIONS

A. General Considerations

Devices of the invention can be formulated using routine methods. All that is required is determination of the desired final concentration of osteogenic protein per device, keeping in mind that the delivered volume of device can be, but is not necessarily required to be, less than the volume at the defect site. Useful formulation methodologies include lyophilization of solubilized protein onto a calcium phosphate matrix. Useful protein solubilization solutions include acidic ethanol, urea, acidic buffers, and acetonitrile/trifluoroacetic acid solutions, and the like. See, for example, U.S. Pat. No. 5,266,683. The desired final concentration of protein will depend on the specific activity of the protein as well as the type, volume, and/or anatomical location of the defect. Proteins having lower specific activity also can be used to advantage. Additionally, the desired final concentration of protein can depend on the age, sex and/or overall health of the recipient. Typically, for a critical size bone segmental defect approximately at least 2.5 cm in length, 0.5-1.75 mg osteogenic protein has been observed using the standard device to induce bone formation sufficient to repair the gap. In the case of a non-critical size defect or a fresh fracture, approximately 0.1-0.5 mg protein has been observed using the standard osteogenic device to repair the defect. Optimization of dosages requires no more than routine experimentation and is within the skill level of one of ordinary skill in the art.

Osteogenic devices and formulations are readily sterilized using standard procedures prior to implantation. For example, proteins conveniently can be filter-sterilized, e.g., using a 0.22 micron filter. Matrix and/or carrier materials can be sterilized by exposure to chemicals, heat, or ionizing radiation. In addition, osteogenic devices and formulations can be terminally sterilized to a sterility assurance level of 10⁻⁶ by exposure to ionizing radiation, for example, gamma or electron beam radiation. Useful dose ranges include within the range of about 0.5-4.0 megarads, preferably 2.0-3.5 megarads. See, for example, U.S. Ser. No. 08/478,452 filed Jun. 7, 1995, or WO 96/40297.

Practice of the invention will be still more fully understood from the following examples, which are presented herein for illustration only and should not be construed as limiting the invention in any way.

### III. BIOASSAY

A. Bioassay of Osteogenic Activity: Endochondral Bone Formation and Related Properties

The art-recognized bioassay for bone induction described by Sampath and Reddi (Proc. Natl. Acad. Sci. USA (1983) 80:6591-6595) and U.S. Pat. No. 4,968,590, the disclosures of which are incorporated by reference herein, are useful to establish the efficacy of a given device or formulation. Briefly, the assay consists of depositing test samples in subcutaneous sites in recipient rats under ether anesthesia. A vertical incision (1 cm) is made under sterile conditions in the skin over the thoracic region, and a pocket is prepared by blunt dissection. In certain circumstances, approximately 25 mg of the test sample is implanted deep into the pocket and the incision is closed with a metallic skin clip. The heterotropic site allows for the study of bone induction without the possible ambiguities resulting from the use of orthotopic sites.

The sequential cellular reactions occurring at the heterotropic site are complex. The multistep cascade of endochondral bone formation includes: binding of fibrin and fibronectin to implanted matrix, chemotaxis of cells, proliferation of fibroblasts, differentiation into chondroblasts, cartilage formation, vascular invasion, bone formation, remodeling, and bone marrow differentiation.

Successful implants exhibit a controlled progression through the stages of protein-induced endochondral bone development including: (1) transient infiltration by polymorphonuclear leukocytes on about day one; (2) mesenchymal cell migration and proliferation on about days two and three; (3) chondrocyte appearance on about days five and six; (4) cartilage matrix formation on about day seven; (5) cartilage calcification on about day eight; (6) vascular invasion, appearance of osteoblasts, and formation of new bone on about days nine and ten; (7) appearance of osteoblastic and bone remodeling on about days twelve to eighteen; and (8) hematopoietic bone marrow differentiation in the ossicle on about day twenty-one. The timecourse of this process varies according to the matrix

Histological sectioning and staining is preferred to determine the extent of osteogenesis in the implants. Staining with toluidine blue or hemotoxylin/eosin clearly demonstrates the ultimate development of endochondral bone. Twelve day bioassays are sufficient to determine whether bone inducing activity is associated with the test sample.

Additionally, alkaline phosphatase activity can be used as a marker for osteogenesis. The enzyme activity can be determined spectrophotometrically after homogenization of the excised test material. The activity peaks at 9-10 days in vivo and thereafter slowly declines. Samples showing no bone development by histology should have no alkaline phosphatase activity under these assay conditions. The assay is useful for quantitation and obtaining an estimate of bone formation very quickly after the test samples are removed from the rat. For example, samples containing osteogenic protein at several levels of purity have been tested to determine the most effective dose/purity level, in order to seek a formulation which could be produced on an industrial scale. The results as measured by alkaline phosphatase activity level and histological evaluation can be represented as “bone forming units”. One bone forming unit represents the amount of protein that is needed for half maximal bone forming activity on day 12. Additionally, dose curves can be constructed for bone inducing activity in vivo at each step of a purification scheme by assaying various concentrations of protein. Accordingly, the skilled artisan can construct representative dose curves using only routine experimentation.

B. Methods of Using a Hydroxyapatite Matrix for Delivery of Osteogenic Protein

An osteogenic device for use in methods of the present invention may comprise any combination of materials suitable to simulate bone growth. Ideally, such materials comprise a biocompatible matrix, implanted at the defect site, upon which new bone growth occurs; an osteogenic protein to stimulate optimal bone growth; and a concentration of calcium phosphate (e.g., hydroxyapatite) to modulate uniform ingrowth. For example, a preferred osteogenic device for use in methods according to the present invention comprises a bovine bone collagen matrix, a ceramic matrix, or a ceramic-collagen composite matrix. The matrix ideally is absorbed into new bone as bone formation takes place. The contents of the matrix may be varied in order to suit a desired clinical application. For example, the source of the matrix may be varied, as may the osteogenic protein used. Numerous sources of bone matrix (e.g., bovine bone, human bone, collagen, and composites) are known. See, e.g., U.S. Pat. Nos. 4,975,526, and 5,354,557, each of which is incorporated by reference herein.

Hydroxyapatite is made by reacting Tetracalcium phosphate and dicalcium phosphate anhydrous or dicalcium phosphate dihydrate in aqueous solution. Hydration of the reactants causes the cement to harden within about thirty minutes to form a microcrystalline lattice. Hydroxyapatite alone will be converted to bone when implanted in physical contact with existing bone near a defect site. However, when osteogenic protein is added, new bone growth, with concomitant osteoconversion of hydroxyapatite, occurs even when there is no physical contact with existing bone. Osteogenic protein generally facilitates the osetoresein on hyroxyapatite-based implants. Methods for preparing hydroxyapatite are reported, inter alia, in U.S. Pat. Nos. Re. 33,161 and Re. 33,221, each of which is incorporated by reference herein.

Improved methods according to the present invention are especially useful in the repair of craniofacial damage or defects, or in other cosmetic surgery applications in which a uniform appearance of new bone is desired. For example, maxillary and mandibular atrophy is a recurrent problem in maxillofacial surgery. It is often difficult to obtain both the required functional results and to preserve aesthetic quality. As shown below, the amount and quality of bone formation in these bones maybe affected by the choice of matrix.

The effects of hydroxyapatite on osteogenic protein induction of new bone growth were analyzed in a mandibular augmentation procedure. Bilateral pouches were created between the lateral aspect of the mandible and the masseteric muscle in 30 male Wistar rats. The bone surface was penetrated to induce bleeding, and one of six different carriers, each containing recombinant OP-1, were delivered to the defect site. The first two carriers were commercially-available hydroxyapatite matrices (Algipore® and Bio-Oss® ). Those were compared with commercially available preparations consisting of silicon granules (Ionogran® ), bovine bone matrix (Osteovit® ), or a collagen bone matrix. In each case, about 70 μg human recombinant OP-1 in acetate buffer was applied to the matrix. The contralateral side was used as a control in which the matrix contained only buffer.

Sequential post-operative labeling of the implant with fluorochromes was performed to enable visualization of bone ingrowth. The animals then were sacrificed at post-operative day 50, and undecalcified ground sections were processed for microradiography, fluorescent light evaluation, and histo-morphometry. Among the variables assessed were height of augmentation and amount of newly-formed bone. Only negligible bone apposition to the underlying mandibular bone was observed in control sites. While augmentation was observed in all OP-1 implanted sites, significant variations in structure and amount of newly-formed bone were found, depending on the matrix that was used. Results are presented in the table below. Mean augmentation height is provided in mm +/−SD. Bone density was determined as the area of newly-formed mineralized bone per total area of augmentation on 3 microradiographs from each specimen.

| | Mean Augmentation | |
|---|---|---|
| Matrix | Height (mm) | Bone Density |
| Algipore ® | 4.1 ± 0.8 | 51% |
| Bio-Oss ® granules | 5.2 ± 1.3 | 27% |
| Bio-Oss Spongiossa block ® | 5.1 ± 0.9 | 22% |
| Osteovit ® | 4.9 ± 1.2 | 42% |
| Ionogran ® | 3.2 ± 0.7 | 9.0% |
| Collagen Bone Matrix ® | 1.0 ± 0.9 | 84% |

As shown in the table, augmentation height was significantly lower when either Ionogran® or collagen bone matrix was used as the matrix. The optimal combination of a desirable augmentation height and optimal density for cosmetic purposes was observed only in cases in which a hydroxyapatite matrix was used.

### IV. FORMULATION OF OP-1/HA COMPOSITE DEVICES

A rat subcutaneous model was used to evaluate the clinical effectiveness of osteogenic protein/hydroxyapatite composite devices containing an optimal ratio of osteogenic protein to hydroxyapatite. A preferred osteogenic protein for use in methods of the invention is the osteogenic protein, OP-1. However, any osteogenic protein may be used in the manner taught herein.

Hydroxyapatite-based matrix formulations may take numerous forms. For example, they may be liquid solutions (e.g. 5% lactose, 20 mM acetate/5% mannitol, pH 4.5), liquid gels (e.g. 5% CMC, 1% alginate), or putty pastes (e.g. CMC powder, gelatin powder). Porous hydroxyapatite blocks (discs) are preferred. Also preferred is a putty device comprising collagen matrix powders with Osteonics granules (0.6 g CMC: 1.0 g HA). Bone formation was inhibited at low doses (<5 μg), and was equivalent to controls at larger doses (>10 μg). Also preferred are synthetic devices comprising 50% HA and 50% tricalcium phosphate.

A preferred OP-1 hydroxyapatite (HA) device was prepared by dry mixing OP-1 in a bone collagen device and HA in a ratio of 1:600 (OP-1/HA). The desired ratio of OP-1 to HA was achieved by mixing 2.5 mg OP-1 per gram of matrix, and then adding HA in a ratio of 60:40 (weight per weight) with respect to the matrix. The matrix was a bovine bone matrix, essentially as disclosed in U.S. Pat. No. 5,354,557, incorporated by reference herein. The OP-1 was formulated essentially as disclosed in U.S. Pat. No. 5,324,819, incorporated by reference herein.

Bilateral Subcutaneous pockets were created in the thorax of rats according to the protocol described in, incorporated by reference herein. A 25 mg aliquot comprising 10 mg of the OP-1 device (25 μg OP-1) was placed into each subcutaneous pocket. The rats were then sacrificed on day 3, 7, 12, 21, or 35. Implants were removed after sacrifice and fixed and demineralized in Boin's solution. The samples were then embedded in paraffin, sectioned and stained with Toluidine Blue.

About 90% of the OP-1/HA composite device comprising an optimal ratio (1:600) of OP-1 to HAC remained at day 7, with significant osteoconversion only on the periphery of the defect site. The time course for HA/OP-1 composite implants revealed a subsequent gradual conversion of the implant to endochondrial bone. Bone growth increased substantially by day 12 (50% of composite remaining) and day 21(20% of composite remaining). There is no residual HA at day 35 post-implantation, and new bone growth is uniform in the defect site, with no evidence of annulus formation or the formation of any other non-uniform growth patterns.

When the OP-1 device was implanted without HA, the matrix disappeared at about day 21 and good bone and marrow were observed, but there was evidence of annulus formation at the periphery of the ingrowth. HA disks coated with OP-1 produced no new bone growth after 12 days post-implantation.

The foregoing results demonstrate that HA/OP-1 composite devices incorporating an optimal ratio of osteogenic protein to hydroxyapatite produce uniform ingrowth of new bone, while ensuring complete resorption of HA.

A. Comparison of HA Materials

Two hydroxyapatite ceramic materials obtained from were evaluated as vehicles for delivery of an HA-OP-1 device for bone repair. Both materials were made from the same hydroxyapatite starting material, but one was sintered at 850 degrees Celsius and the other at 800 degrees Celsius. Both materials were in particle form with diameters ranging from about 212 μm to about 425 μm. Each of the hydroxyapatite materials was combined with varying amounts of OP-1 and the composites were evaluated for their ability to stimulate bone formation in rat subcutaneous sites. In general, about 60 mg of these hydroxyapatite occupies the same volume as about 25 mg of collagen.

Subcutaneous implants were made in rats as described above. Generally, bone formed into the implants with 10 μg of OP-1 per 60 mg HA.

The rate of release of OP-1 from collagen and hydroxyapatite into serum was next compared. About 62.5 μg of OP-1 was formulated with 25 mg of collagen or 60 mg of hydroxyapatite in 47.5% ethanol and 0.1% triflouroacetic acid. After lyophilization, the devices were transferred to clean tubes and incubated with 1 ml of serum at 37 degrees Celsius. The serum was removed at the designated time points and replaced with fresh serum. Serum OP-1 levels were quantified by ELISA. It was determined that OP-1 release was similar from both the collagen and hydroxyapatite materials.

B. Effect of Co-Lyophilization of Op-1 and HA

Studies were done to assess the efficacy of implants comprising hydroxyapatite which had been co-lyophilized with OP-1 compared to a non-lyophilized formulation comprising hydroxyapatite particles mixed in an OP-1 solution comprising, in addition to OP-1, 20 mM acetate, pH 4.5, and 5% mannitol.

It was determined that either of the above formulations support adequate bone growth when 5 μg OP-1 was combined in 60 mg hydroxyapatite.

Next, the compatibility of OP-1 and hydroxyapatite particles sterilized by gamma radiation and the stability of an OP-1/HA composite sterilized by gamma radiation were evaluated.

An OP-1/HA device was formulated from 60 mg HA with 60 μg OP-1 by co-lyophilization from 47.5% ethanol/0.01% triflouroacetic acid. The formulation was sterilized using 2.5-3.0 mrads of gamma radiation. A control device (no irradiation) was also made. The irradiated and control devices were eluted with Urea buffer, and analyzed by reverse phase HPLC. Approximately 30-40% of the OP-1 was lost upon irradiation, which is typical of the amount of loss experienced when the collagen device is used. A Ross cell assay indicated that extracted OP-1 retained its biological activity.

### V. CAT CRANIAL DEFECT MODEL

An OP-1 bovine collagen device was mixed with hydroxyapatite cement HAC, wherein human osteogenic protein (OP-1) and HAC exist in a ratio of about 1:600 (OP-1: HAC) and was used to repair induced cranial defects in cats. The composite device was essentially the same as the 1:600 device described above in Example 1. An OP-1/HAC collagen composite device was compared to a control of pure HAC three months after implant using gross examination, computed tomography, and histologic/histometric techniques.

Devices were implanted in the cats as follows. The parietal skull was exposed bilaterally after a midline scalp incision, and the periosteal layer was separated from the skull as a flap. Using a high-speed cutting burr, two full-thickness craniotomies were created on the parietal skull, each about 2.5 cm in diameter, and symmetrical on either side of the midline. In each cat, pure HAC was placed into the area of the right craniotomy defect; whereas the left defect was replaced by the OP-1/HAC collagen device. After shaping of the external contour, implants were allowed to solidify and the incisions were closed in layers.

There were no wound infections, implant infections, or implant extrusions in any cat. The were sacrificed at 3 months post-operative. Skulls were removed and subsequently examined visually for shape, contour and overall appearance. The skulls were then examined by computed tomography with a bone algorithm in order to visualize the implanted areas. Next, the entire outer calvarial portion of the skull, containing both implanted areas was removed and embedded in methylmethacrylate for undecalcifed whole sections. Paragon- and Von Kossa-stained sections were obtained in order to differentiate HAC, bone, and osteoid components of bone. Histometric analysis was performed to determine the volume fraction of tissue components at the central area of the implant, the implantibone interface, and the normal calvarial bone. In addition, the percent HAC resorption/replacement was determined by measuring the relative area of remnant implant over the entire implanted area.

A. Gross Histologic Examination

Upon visual examination of the removed skulls, the pure HAC implant appeared intact with shape, contour, and volume very close to that of the initially-applied cement. New bone ingrowth occurred only on the periphery of the defect site. The HAC implant was visible as a lighter color than the surrounding bone and was well-integrated with the surrounding bone. No volume change was apparent in the area of the implant.

In contrast, the OP-1/HAC collagen composite device implant was fully-replaced by new bone. There was no evidence of any remaining implant from the external surface or from the internal surfaces of the calcium. The shape, volume, and contour of the implanted area was preserved in the new bone.

B. Cross-Sectional Imaging

Coronal computerized tomography images of removed skulls revealed full-thickness, stable HAC implant on the HAC (right) side, with some peripheral induction of new bone. On the HAC/OP-1 device side, there was nearly-complete conversion of the composite implant, with the appearance of new lamellar bone. Again, the volume and shape of the new bone was well-preserved.

C. Histology

Paragon and Von Kossa staining revealed growth of new dense bone on the HAC/OP-1 side. The majority of the implant had been replaced by bone. Some remnant implant was seen as dark particles at the center of the implant. By measuring the relative cross-sectional area of the implant versus bone over the entire implanted area, it was calculated that about 93% (SD 3.7) of the composite implant was resorbed and replaced by new bone.

The foregoing data indicate that the composite implant of HAC/OP-1 accelerates the process of implant resorption and new bone formation. Pure HAC was successful only in reconstructing the cranial defect with ostoeintegration on the periphery of the implant site.

### VI. USE OF HA/OP-1 DEVICE TO REPAIR BABOON CRANIAL DEFECTS

The efficacy of a single application of various doses of OP-1 absorbed onto sintered hydroxyapatite-based ceramic porous shaped form for osteointegration and bone regeneration in calvarial defects of adult baboons was next determined.

Sintered porous ceramic vehicles were made by heating hydroxyapatite slurries in stages and then sintering. The resulting material was a disc of porous sintered ceramic of about 25 mm in diameter and about 4 mm in thickness. Animals used in the experiments were four clinically-healthy adult male Chacea baboons (Papio ursinus). The mean weight of all subjects was 28.7 kg (+/−2.3). Each subject had normal hematologic and biochemical profiles. The animals were obtained from the primate colony at Witwatersrand, Johannesburg, South Africa.

OP-1 was prepared by dissolving 0.0 (5 mM HCl control), 100, or 500 μg OP-1 in 500 μl of 5 mM Hcl. Sintered ceramic discs were prepared as described above. Absorption of the OP-1 into the ceramic carrier was carried out under sterile conditions. The OP-1 solutions were pipetted into the discs and air-dried.

Cranial defects were prepared in each of the animals using a craniotome. Two full-thickness defects, each about 25 mm in diameter, were prepared on each side of the calvaria. The defects were separated by about 3 cm of intervening calvarial bone. In each animal, 2 defects were implanted with sintered discs pretreated with 100 μg OP-1; one defect was treated with a device comprising 500 μg OP-1; and the fourth defect was implanted with a sintered porous disc with no OP-1 (control). Four weeks after implantation, the animals were sacrificed with an intravenous dose of pentobarbitone. Specimen blocks were cut along the sagittal third of the implanted discs and fixed in 10% neutral buffered formaldehyde. Specimens were then decalcified in a formic-hydrochloric acid mixture. Serial sections, each about 5 μm thick, were mounted after recording the position of the anterior and posterior interfaces of the defects with their corresponding calvarial margins. Sections were stained with Goldmer's trichome or with 0.1% toluidine blue in 30% ethanol. A calibrated Zeiss Integration Platte II (Zeiss) with 100 lattice points was used to calculate, by point counting techniques, the fractional volume (in percent) of the newly-formed bone. Sections were analyzed at a magnification of 40 times with a Zeiss graticule superimposed over five sources selected for histomorphometry and defined as follows: two anterior and posterior interfacial regions (AIF and PIF, respectively) two anterior and posterior internal regions (AIN and PIN, respectively) and a control region (CON). This technique allows the histomorphometric evaluation of the distribution of bone deposition across the hydroxyapatite susbstrata. Each source represented a field of 7.84 mm². Morphometry was performed on two sections per specimen, representing parasagittal levels approximately 5 mm apart from each other.

At sacrifice, control specimens showed fibrovascular tissue invasion across the porous spaces of the ceramic device, with some bone formation at the edges of the calvarial defects. There was no bone formation within the central or internal regions of the specimens. No significant resorption of the ceramic occurred in the control.

Porous ceramics pretreated with 100 μg OP-1 showed extensive bone formation within the porous spaces and in direct apposition with the substratum. There was prominent vascular invasion and the newly-formed bone had the features of trabecular woven bone extending into the porous spaces. Moreover, there was complete incorporation of the ceramic disc by newly-formed bone within the severed calvarial bone. Porous ceramics pretreated with 500 μg OP-1 showed bone formation only on the endocranial and pericranial aspects of the specimen, enveloping the ceramic substratum. The internal porous spaces of the ceramics were characterized by the presence of a rich vascular component, but bone formation was not observed. With both the 100 μg and the 500 μg samples, significant resorption of the ceramic material was observed when compared with the control implants. The results of histomorphic analysis of the samples described above are shown in Table 2 below; wherein values are means of four control specimens and 12 OP-1 treated porous HA specimens.

TABLE 2

| Treatment | Control | 100 μg OP-1 | 500 μg OP-1 |
|---|---|---|---|
| Volume fraction of | 0.7 | 32.6 | 23.6 |
| induced bone (%) | | | |

The foregoing results indicate that the use of a ceramic carrier for high concentrations of OP-1 in the absence of a collagenous carrier results in bone formation mainly on the perennial and endocranial aspects of the specimens. With 500 μg OP-1, bone formation mainly enveloped the carrier and was rarely in contact with the substratum. In contrast, use of lower relative amounts of OP-1 (in the range of less than about 1:100 with respect to HAC) resulted in full, uniform induction of new bone.

### VII. METAL IMPLANTS FOR GAP DEFECT REPAIR

In other embodiments of the invention, bone ingrowth in a defect is stimulated by implanting a metal implant in a bed of OP devices at the defect site. For example, a defect site, prepared by removing excess or necrotic bone tissue, is filled with an OP device. A metal implant, preferably coated with hydoxyapatite is then placed in the defect so that the OP device fills gap between the metal implant and the edges of the defect site. Preferably, and OP device comprises a morphogen, as herein described, in a suitable adjuvant, or in a collagen matrix, or another matrix as described herein. the following are several examples demonstrating this process.

A. Repair of gap defects using metal implant allograft therapy

Methods of the invention comprise repair of impaired bone stock, in, for example, revision endoprosthetic surgery, using impacted allograft bone chips. Adding the osteoinductive stimulation of osteogenic protein 1 (OP-1) to the osteoconductive effects of the allograft bone chips improves clinical outcome. OP-1 mixed into impacted allograft to improve bone formation and mechanical fixation of hydroxyapatite coated implants.

The effects of the OP-1 in Impacted allograft was evaluated in a canine model. Cylindrical hydroxyapatite-coated titanium alloy implants with an edged surface texture were used. The implants measured 4 ×9 mm and were inserted unloaded and bilaterally into the proximal humerus of 16 adult mongrel dogs surrounded by a 3 mm gap. Two different doses were tested in 8 animals each: (1) 325 μg OP-1 in 130 mg collagen matrix mixed into 1.3 g of allograft chips. (2) 65 μg OP-1 in 130 mg collagen matrix mixed into 1.3 g of allograft chips. The contralateral humerus were used for control in which the allograft were mixed with collagen matrix without OP-1 only. The dogs were sacrificed after 6 weeks. Bone ingrowth and bone formation in the gap was evaluated by quantitative histomophometry and mechanical fixation of the implants were evaluated by push-out test.

Histomorphometry demonstrated an increase in pan-implant bone volume from 25.5% to 33.5% of total peri-implant gap volume. This increase was found for the 65 μg dose but not for the 325 μg dose. Bone ingrowth was relatively high for control groups (about 35%) and was not altered by OP-1 addition to the allograft. mechanical testing showed that sheer strength, stiffness and energy absorption were not increased for OP-1 simulated groups. These data study demonstrate that the addition of OP-1 to impacted allograft increases bone formation around the implants using the lowest of the two doses tested. Mechanical fixation of the implants was not improved by OP-1 addition which could be explained by a similar lack of increased bone ingrowth in the groups receiving OP-1. These data indicate a clinical use of OP-1 for stimulation of bone formation in revision endoprosthetic surgery.

B. OP device in prosthetic surgery

In cementless endoprosthetic surgery gaps around implants impair bony fixation and clinical outcome. The following study shows that stimulation of bone healing with osteogenic factors improves clinical outcome of osteogenic prostheses.

Osteogenic protein (OP-1) has previously shown extensive in vivo osteoinductive properties in bone defect models, fracture models, and spine fusion models. However, little is known about morphogen's ability to enhance bony fixation of implants. Previously, only TGF-B has demonstrated stimulatory effects on bony fixation and bone formation when applied onto ceramic coated implants surrounded by a gap. The purpose of the present study was to determine if mechanical fixation of uncoated and hydroxyapatite coated implants is enhanced by applying OP-1 in a collagen carrier in a critical-sized, 3 mm, gap around the implants.

Enhancement of implant fixation with recombinant OP-1 was evaluated in a canine model approved by both the Danish and Stanford University control board for animal research. Cylindrical uncoated and hydroxyapatite coated titanium alloy implant (Ti6AI4V) (4×10 mm) with an edged surface texture were used. The implants were inserted unloaded bilaterally into both the medial and the lateral femoral condyles of 26 adult mongrel dogs. All implants were initially surrounded by a 3 mm gap. Eight dogs were included in each implant group. Three different groups for each type of implant were tested. (1) OP-1 device (325 μg OP-1 in 130 mg collagen matrix (2) The collagen matrix without Op-1; (3) Empty gap. The OP-1 device and the collagen carrier was placed in the gap around the implants. The dogs were sacrificed after 8 weeks.

After sacrifice, mechanical fixation of the implants were evaluated by push-out test of a 3.5 mm thick specimen on an Instron universal testing machine. Shear strength, sheer stiffness and energy absorption was determined. One way ANOVA and paired Students T-test were used. P values less than 0.05 were considered significant.

All dogs completed the study. Mechanical testing showed that shear strength, stiffness and energy absorption were significantly increased for OP-1 stimulated significant increased values above both control and collagen groups with respect to strength and stiffness, whereas for energy absorption OP-1 was only significantly different from the control group. For HA coated implants, the OP-1 group was significantly higher than both the control and collagen groups with respect to stiffness. Whereas for strength and energy absorption the OP-1 group was only significantly different from the control group. Collagen matrix was significantly higher that empty control for both implant types. The present study demonstrates that the addition of OP-1 device increases mechanical fixation of both uncoated and hydroxyapatite coated implants. The strongest effects were found for uncoated implants where OP-1 device stimulated fixation to the same level as hydroxyapatite coated implants stimulated with the OP-1 device, since the empty control group no fixation was found. Interestingly, the collagen matrix by itself demonstrated a considerable stimulatory effect on fixation, probably due to an osteoconductive effect; whereas the collagen matrix captures stem-cells and other growth factors that will improve bone formation in the gap. The collagen matrix that results also emphasizes the importance of including such carrier controls in studies investigating systems and stimulative agents. For hydroxyapatite coated implants these effects were so dominant that no significant increase was demonstrated for hydroxyapatite coated implants with OP-1 device compared to hydroxyapatite coated implants with the collagen carrier alone, although a 40% increase was found. The study also showed that hydroxyapatite coating alone can bridge a gap of 3 mm in 6 weeks to provide some fixation, but that adding the OP-device strongly enhances bone formation to provide implant fixation in the range of 3 MPa regardless of whether uncoated or HA coated implants are used. These data are interesting for the use of OP-1 for stimulation of bony ingrowth in primary cements endoprosthetic surgery since OP-1 device was shown to enhance mechanical strength for the bone-implant interface of both uncoated and HA coated implants.

C. Hydroxyapatite-coated metal implants

Early incorporation of bone allograft material is of great importance. OP-1 (BMP-7) further enhances bone incorporation around implants, and Pro-Osteon 200 is an alternative to bone allograft.

The purpose of this study was to investigate whether OP-1 device in combination with bone allograft improves the early fixation of implants compared to the allograft alone. We also compared allograft to Pro-Osteon with and without OP-1.

Unloaded cylindrical titanium implants (5-10 mm) coated with hydroxyapatite were randomly inserted in the medial and lateral femoral condyles of 6 labrador dogs described in Sabelle, et al. Clin. Ortho. 274: 283-293 (1992), incorporated by reference herein. A cavity of 11 mm in diameter was created by hand-drilling leaving a gap of 3 mm (0.75 cc volume) around the implant. The implant was secured by a footplate and after grafting the gap, a top washer and a screw.

The gaps around the implants were filled according to the following treatment groups: Group 1: Allograft, group 2 Pro-Osteon, group 3: Allograft+OP-1 device, group 4: Pro-Osteon+OP-1 device. Allograft was harvested from a dog not included in the study, frozen at −80° C., thawed and milled in an operating room with laminar air flow. The amount of Pro-Osteon and allograft was standardized by weight. OP-1 was delivered in a device with 2.5 mg recombinant OP-1 in 1 gram bovine type 1 collagen, the concentration of OP-1 in the present study was 300 μg Op-1 in 120 mg collagen carrier. The bone substitute utilized was Pro-Osteon 200 (Interpore, Irvine U.S.) corraline hydroxyapatite granules which has a porous microstructure similar to cortical bone. This product is a non-osteogenic void filler approved by the FDA for metaphyseal defects.

Dogs were sacrificed after 3 weeks and push-out and mechanical fixation was evaluated by a push-out test performed on and Instron Universal testing machine. Ultimate shear strength (MPa), apparent shear stiffness (MPa/mm) and energy absorption (3/mm²) were determined blindly from load displacement curves.

Statistial data are presented as mean values and standard error of mean. An unpaired t-test was used. (P-values less than 0.05 (two-tailed) were considered significant.) Push-out tests showed, that the OP-1 device enhanced fixation of Pro-Osteon by 900%. No significant differences were found between Pro-Osteon +OP-1 and allograft with or without OP-1, but all three groups had significantly better mechanical parameters compared to the Pro-Osteon group.

Implants treated with bone allograft were much stronger fixated than implants treated with Pro-Osteon alone. However in combination with OP-1, Pro-osteon treated implants were equally fixed compared to the allograft group. The fact the OP-1did not improve the fixation of the allograft group indicates that the allograft is a superior gap filling material around non cemented implants compared to Pro-Osteon. on the other hand, a bone substitute (pro-Osteon) in combination (OP-1) seems to be just as good as bone allograft.

Early incorporation of bank bone allograft is mandatory in revision of anthroplasties. We investigate, if OP-1 (BMP-7) device further enhances bone incorporation around implants and if Pro-Osteon 200 might be an alternative to bone allograft.

24 unloaded cylindrical titanium implants coated with hydroxyapatite were randomly inserted in the femoral condyles of 6 labrador dogs. A 3 mm gap was left around each implant, which were filled according to the following groups: (1) Allograft, (2) Pro-Osteon, (3): Allograft+OP-1, (4) Pro-Osteon+OP-1. the amount of Pro-Osteon, OP-1 and allograft was standardized by weight.

Push out tests after 3 weeks showed, that the OP-1 enhanced fixation of Pro-Osteon by 900%. No significant differences were found between Pro-Osteon+Op-1 and allograft with or without OP-1. The study showed, that implants treated with bone allograft were much stronger fixated than implants treated with Pro-Osteon alone, however in combination with OP-1, Pro-Osteon treated implants were equally fixed compared to the allograft group.

What is claimed is:

1. A method for producing new bone growth at bone defect site in a mammal, the method comprising the step of:
implanting in a defect site a calcium phosphate matrix comprising at least one osteogenic protein.

2. The method of claim 1, wherein said calcium phosphate matrix is a hydroxyapatite matrix.

3. The method of claim 1, wherein said osteogenic protein is a dimeric protein that comprises an amino acid sequence selected from the group consisting of:
(a) a sequence having at least 70% homology with the C-terminal seven-cysteine skeleton of human OP-1, residues 38-139 of SEQ ID NO: 5, and
(b) Generic Sequence 6, SEQ ID NO: 31; and
wherein said morphogen stimulates endochondral bone formation in an in vivo bone assay.

4. The method of claim 1, wherein said osteogenic protein is a dimeric protein that comprises an amino acid sequence selected from the group consisting of:
(a) a sequence having greater than 60% amino acid sequence identity with the C-terminal seven-cysteine skeleton of human OP-1, residues 38-139 of SEQ ID NO: 5, and
(b) OPX sequence defined by SEQ. ID No: 29; and
wherein said osteogenic protein stimulates endochondral bone formation in an in vivo bone assay.

5. The method of claim 1, wherein said osteogenic protein is selected from the group consisting of human OP-1, mouse OP-1, human OP-2, mouse OP-2, 60A, GDF-1, BMP2A, BMP2B, DPP, Vg1, Vgr-1, BMP3, BMP5, and BMP6.

6. The method of claim 1, wherein said osteogenic protein is a conservative substitution variant of a morphogen selected from the group consisting of human OP-1, mouse OP-1, human OP-2, mouse OP-2, 60A, GDF-1, BMP2A, BMP2B, DPP, Vg1, Vgr-1, BMP3, BMP5, and BMP6.

7. A method for producing new bone growth at a defect site in a mammal, the method comprising the step of:
implanting an osteogenic device in said defect site, the osteogenic device comprising an osteogenic protein and a biocompatible matrix; wherein said biocompatible matrix comprises calcium phosphate in an amount such that the ratio of calcium phosphate to said osteogenic protein is sufficient to produce uniform ingrowth of new bone in said defect site.

8. The method of claim 7, wherein said calcium phosphate is hydroxyapatite.

9. The method of claim 7, wherein said ratio of calcium phosphate to osteogenic protein is less than about 1:10,000.

10. The method of claim 7, wherein said ratio of calcium phosphate to osteogenic protein is about 1:2000.

11. The method of claim 7, wherein said ratio is about 1:600.

12. The method according to claim 7, wherein said ratio is 1:700.

13. The method according to claim 7, wherein said ratio is 1:500.

14. The method according to claim 7, wherein said ratio is 1:1000.

15. A method for stimulating new bone growth at a defect site in a mammalian bone, comprising the step of:
introducing calcium phosphate and osteogenic protein to a defect site in a ratio sufficient to produce uniform ingrowth of new bone.

16. The method of claim 15, wherein said calcium phosphate is hydroxyapatite.

17. The method of claim 16, wherein said hydroxyapatite is a sintered hydroxyapatite.

18. The method of claim 15, wherein said ratio is less than about 1:100.

19. The method of claim 15, wherein said ratio is about 1:500

20. The method of claim 15, wherein said ratio is about 1:600.

21. The method of claim 15, wherein said ratio is about 1:700.

22. A method for inducing uniform calcium resorption in a bone defect site, the method comprising the steps of:
implanting in said defect site an osteogenic device comprising a biocompatible matrix, calcium phosphate, and an osteogenic protein; wherein said osteogenic protein and said calcium phosphate are present in said device in a ratio of less than about 1:1000.

23. The method of claim 22, wherein said calcium phosphate is hydroxyapatite.

24. The method of claim 22, wherein said ratio is about 1:500.

25. The method of claim 22, wherein said ratio is about 1:600.

26. The method of claim 22, wherein said ratio is about 1:700.

27. A method for promoting bone ingrowth in a defect site, the method comprising the steps of:
(a) implanting in a defect site a metal implant; and
(b) surrounding said implant with a composition comprising a morphogen selected for the groups consisting of:
(1) a morphogen having at least 70% amino acid homology with the C-terminal, seven-cysteine domain of human OP-1, SEQ ID NO: and
(2) a morphogen having at least 60% amino acid identity with the C-terminal, seven-cysteine domain of human OP-1, SEQ ID NO: .

28. The method of claim 27, wherein said metal implant is a titanium implant.

29. The method of claim 27, wherein said metal implant is coated with hydroxyapatite.

30. The method of claim 27, wherein said morphogen is OP-1.

31. The method of claim 27, wherein said morphogen is selected from the group consists of BMP2, BMP4, BMP5, and BMP6.

32. The method of claim 27, wherein said composition further comprises a collagen matrix.

33. The method of claim 27, wherein said composition further comprises a carboxy methyl cellulose matrix.

34. The method of claim 27 wherein said composition further comprises a calcium phosphate matrix.

35. The method of claim 34, wherein said calcium phosphate matrix is a hydoxyapatite matrix.
